(19) United States
(12) Patent Application Publication
(10) Pub. No.: US 2004/0192784 A1
(43) Pub. Date: Sep. 30, 2004

(54) Combination of a betablocker and a cholesterol-lowering agent

(75) Inventors: Goran Bondjers, Lerum (SE); Olov Wiklund, Molndal (SE); John Wikstrand, Molndal (SE)

Correspondence Address: ROPES & GRAY LLP, ONE INTERNATIONAL PLACE, BOSTON, MA, 02110-2624, US

(73) Assignee: AstraZeneca AB, Sodertalje (SE)

(21) Appl. No.: 10/824,170

(22) Filed: Apr. 14, 2004

Related U.S. Application Data
(63) Continuation of application No. 10/220,790, filed on Sep. 4, 2002, now abandoned.
Application No. 10/220,790 is a 371 of international application No. PCT/SE01/00663, filed on Mar. 27, 2001.

(30) Foreign Application Priority Data
Apr. 3, 2000 (SE) 0001188-2
Jun. 22, 2000 (SE) 0002352-3

(51) Int. Cl. A61K 31/137

(52) U.S. Cl. 514/651

(57) ABSTRACT

The present invention relates to pharmaceutical formulations comprising a betablocker and a cholesterol-lowering agent in admixture with a pharmaceutically acceptable adjuvant, diluent or carrier, as well as a kit of parts, a method for treatment and use of the formulations for the prophylactic or therapeutic treatment of atherosclerosis, hypercholesterolemia and hyperlipoproteinemia.

### CROSS-REFERENCE TO RELATED APPLICATIONS

This application is a national stage filing under 35 U.S.C. 371 of PCT application PCT/SE01/00663, filed Mar. 27, 2001, which claims priority from Sweden Application Nos. 0001188-2, filed Apr. 3, 2000, and 0002352-3, filed Jun. 22, 2000, the specifications of each of which are incorporated by reference herein. PCT Application PCT/SE01/00663 was published under PCT Article 21(2) in English.

### FIELD OF THE INVENTION

The present invention relates to pharmaceutical formulations comprising a betablocker and a cholesterol-lowering agent in admixture with a pharmaceutically acceptable adjuvant, diluent or carrier, as well as a kit of parts, a method for treatment and use of the formulations for the prophylactic or therapeutic treatment of atherosclerosis, particularly in patients suffering from hyperlipidemia, including hypercholesterolemia.

### BACKGROUND OF THE INVENTION

There is a constant need for new medications to further reduce the risk of atherosclerotic disease. This is especially true for patient populations at a higher risk than the population at large, e.g. patients suffering from hypercholesterolemia and hyperlipoproteinemia.

Various pharmaceuticals, such as the group known as statins, are known to lower the concentration of total serum cholesterol as well as influence the ratio between the concentration of the positive high-density lipid (HDL) cholesterol and the negative low-density lipid (LDL) cholesterol.

Betablockers on the other hand are known to have a positive influence on various cardiovascular disease, primarily hypertension, whereas physicians have generally avoided prescription of betablockers for patients with hypercholesterolemia.

The use of a combination of betablockers and cholesterol-lowering agents for the prophylactic and therapeutic treatment of patients suffering from, or susceptible to, atherosclerosis, hypercholesterolemia or hyperlipoproteinemia has not been disclosed previously.

The administration of combinations of β₁ selective blockers and lipid-lowering drugs to healthy volunteers, in order to observe the effects on fat metabolism, ammonia levels and the perception of effort during exercise, was disclosed in Br. J. Clin Pharmacol 1997 vol 43, no 3 pages 291-300. The combinations studied were 1) metoprolol (controlled release) and fluvastatin 2) metoprolol (controlled release) and bezafibrate 3) atenolol (normal release) and fluvastatin and 4) atenolol (normal release) and bezafibrate. The paper concluded that that these four combinations each caused significant reductions in fat metabolism, increased plasma ammonia concentrations and raised the perception of exercise. Combination 1) had the least adverse effect but the formulation difference was thought to be a significant factor in explaining the differences observed with respect to combination 3).

The effects of a combination of pravastatin and atenolol on hypertensive and hypercholesterolaemic patients was reported in Scand. J. Print Health Care 1999. vol 17 122-127. The conclusion was that the effect of atenolol was not influenced by the concurrent administration of pravastatin and vice versa. However, lifestyle intervention was also a feature of this study and therefore the conclusions which can be drawn from this study are not clear.

A post hoc analysis of patients who had been treated with lovastatin and who were also receiving antihypertensive medication including β₁ adrenergic receptor blockers was reported in Hypertension,1992,Vol.19,3 242. The paper concluded that, subject to a number of limitations, there was no evidence for an attenuation of the lovastsatin-induced changes in lipids and lipoproteins or an alteration in the safety profile of lovastatin when administered concurrently with commonly used antihypertensive agents.

It was concluded in Presse Med Volume 1996, vol 25, no.40, 2013-2016 that the effect of the β₁ adrenergic receptor blocker atenolol was not diminished in combination with pravastatin. However, the effect of pravastatin on lipid metabolism was more favourable when pravastatin was combined with the angiotensin converting enzyme inhibitor captopril rather than with atenolol.

WO98/02357 discloses a carton for carrying pharmaceutically active substances or combinations thereof. One such combination mentioned is the combination of a beta blocker, such as metoprolol or isosorbidmononitrate, and a lipid lowering substance, such as fluvastatin. This application does not disclose any data concerning the effects of such a combination.

WO 99/11260 discloses a combination of atorvastatin and an antihypertensive agent. No data are disclosed in this application.

WO97/38694 discloses a combination of an HMG-CoA reductase (3-hydroxy-3-methylglutaryl coenzyme A reductase) inhibitor in combination with folic acid in combination with a drug selected from a range of other types of drug including beta blockers.

WO 00/38725 discloses combinations of an ileal bile transport inhibitor and a range of other types of drug including antihypertensive drugs for example beta blockers. No data are presented.

### SUMMARY OF THE INVENTION

The present invention relates to pharmaceutical combinations containing a betablocker and a cholesterol-lowering agent, pharmaceutical formulations containing a betablocker and a cholesterol-lowering agent in admixture with a pharmaceutically acceptable adjuvant, diluent or carrier, as well as a method for treatment and use of the formulations for the prophylactic or therapeutic treatment of atherosclerosis, hypercholesterolemia and hyperlipoproteinemia.

The invention further relates to a kit of parts of vessels containing the betablocker and the cholesterol-lowering agent and instructions for the administration of the betablocker and cholesterol-lowering, agent to a patient for which such administration is necessary or advantageous.

The invention also relates to a kit of parts of formulations containing the betablocker and the cholesterol-lowering agent each in admixture with a pharmaceutically acceptable adjuvant, diluent or carrier.

### DETAILED DESCRIPTION OF THE INVENTION

It has surprisingly been found that, compared to the use of a cholesterol-lowering agent as a monotherapy, the present invention comprising a combination of a cholesterol-lowering agent and a beta-blocker has an enhanced anti-atherosclerotic effect, particularly in patients suffering from hypercholesterolemia or hyperlipoproteinemia.

In one aspect, the present invention thus relates to pharmaceutical combinations comprising a betablocker and a HMG-CoA reductase inhibitor wherein the betablocker is selected from the group consisting of: acebutolol, alprenolol, amosulalol, arotinolol, atenolol, befunolol, betaxolol, bevantolol, bisoprolol, bopindolol, bucumolol, bufetolol, bufuralol, bunitrolol, buprandolol, butofilolol, carazolol, carteolol, carvedilol, celiprolol, cetamolol, cloranolol, dilevalol, epanolol, indenolol, labetalol, levobunolol, mepindolol, metipranolol, metoprolol, moprolol, nadolol, nadoxolol, nebivalol, nipradilol, oxprenolol, perbutolol, pindolol, practolol, pronethalol, propranolol, sotalol, sufinalol, talindol, tertatolol, tilisolol, timolol, toliprolol, and xibenolol, and pharmaceutically acceptable salts or solvates thereof, or solvates of such salts; and the HMG-CoA reductase inhibitor is selected from the group consisting of: cerivastatin, itavastatin, lovastatin, mevastatin, nicostatin, nivastatin, and (E)-7-[4(4-fluorophenyl)-6-isopropyl-2-[methyl(methylsulfonyl)amino]-pyrimidin-5-yl](3R,5S)-3,5-dihydroxyhept-6-enoic acid or a pharmaceutically acceptable salt or solvate thereof, or a solvate of such a salt comprising a betablocker and a cholesterol-lowering agent.

In another aspect, the present invention relates to pharmaceutical formulations, comprising: a betablocker and a cholesterol-lowering agent in admixture with a pharmaceutically acceptable adjuvant, diluent or carrier.

A further aspect of the present invention relates to kits of parts comprising:

(i) a vessel containing a betablocker and

(ii) a vessel containing a HMG-CoA reductase inhibitor which is (E)-7-[4-(4-fluorophenyl)-6-isopropyl-2-[methyl(methylsulfonyl)amino)-pyrimidin-5-yl](3R,5S)-3,5-dihydroxyhept-6-enoic acid or a pharmaceutically acceptable salt, especially sodium or calcium, or solvate thereof, or a solvate of such a salt

and instructions for the sequential, separate or simultaneous administration of the betablocker and a HMG-CoA reductase inhibitor to a patient for which such administration is necessary or advantageous.

Another aspect of the invention relates to kits of parts comprising:

(i) a pharmaceutical formulation containing a betablocker in admixture with a pharmaceutically acceptable adjuvant, diluent or carrier; and

(ii) a pharmaceutical formulation containing a cholesterol-lowering agent, in admixture with a pharmaceutically acceptable adjuvant, diluent or carrier;

wherein the betablocker and the cholesterol-lowering agent are each provided in a form that is suitable for administration in conjunction with the other.

By “administration in conjunction with”, we include that respective formulations comprising a betablocker and a cholesterol-lowering agent are administered, simultaneously, separately or sequentially, over the course of treatment of the relevant condition, which condition may be acute or chronic. Particularly, the term includes that the two formulations are administered (optionally repeatedly) sufficiently closely in time for there to be a beneficial effect for the patient, that is greater, over the course of the treatment of the relevant condition, than if either of the two formulations are administered (optionally repeatedly) alone, in the absence of the other formulation, over the same course of treatment. It must, however, be emphasized that betablockers have never been used previously to give an anti-atherosclerotic effect e.g. in patients suffering from hypercholesterolemia or hyperlipoproteinemia. Determination of whether a combination provides a greater beneficial effect in respect of, and over the course of treatment of, a particular condition, will depend upon the condition to be treated or prevented, but may be achieved routinely by the person skilled in the art.

Thus, the term “in conjunction with” includes that one or other of the two formulations may be administered (optionally repeatedly) prior to, after, or at the same time as, administration with the other component. When used in this context, the terms “administered simultaneously” and “administered at the same time as” include that individual doses of a betablocker and a cholesterol-lowering agent are administered within 48 hours, e.g. 24 hours, of each other.

Yet another aspect of the invention relates to methods for prophylactic or therapeutic treatment of a patient suffering from, or susceptible to, atherosclerosis, which method comprises administering to the patient a therapeutically effective total amount of

(i) a betablocker in admixture with a pharmaceutically acceptable adjuvant, diluent or carrier; in conjunction with

(ii) a cholesterol-lowering agent in admixture with a pharmaceutically acceptable adjuvant, diluent or carrier.

Still another aspect of the invention relates to methods for prophylactic or therapeutic treatment of a patient suffering from, or susceptible to, atherosclerosis, which method comprises administering to the patient a pharmaceutical formulation, comprising:

(i) a betablocker and

(ii) a cholesterol-lowering agent in admixture with a pharmaceutically acceptable adjuvant, diluent or carrier.

In a further aspect of the invention relates to methods for prophylactic or therapeutic treatment of a patient suffering from, or susceptible to, atherosclerosis, which method comprises administering to the patient a pharmaceutical combination consisting essentially of

(i) a betablocker and

(ii) a cholesterol-lowering agent.

In another aspect of the invention relates to methods for prophylactic or therapeutic treatment of a patient suffering from, or susceptible to, atherosclerosis, which method comprises administering to the patient a pharmaceutical combination consisting of

(i) a betablocker and

(ii) a cholesterol-lowering agent.

A still further aspect of the invention relates to the use of pharmaceutical formulations, comprising:

(i) a betablocker and

(ii) a cholesterol-lowering agent in admixture with a pharmaceutically acceptable adjuvant, diluent or carrier,

in the manufacture of a medicament for the prophylactic or therapeutic treatment of atherosclerosis.

Betablockers

In the present application, the term “betablockers” refers to any pharmaceutical agent that as part of its pharmacological action blocks beta-one-receptors.

The betablockers referred to in this application include but are not limited to the compounds selected from the group consisting of acebutolol, alprenolol, amosulalol, arotinolol, atenolol, befunolol, betaxolol, bevantolol, bisoprolol, bopindolol, bucumolol, bufetolol, bufuralol, bunitrolol, buprandolol, butofilolol, carazolol, carteolol, carvedilol, celiprolol, cetamolol, cloranolol, dilevalol, epanolol, indenolol, labetalol, levobunolol, mepindolol, metipranolol, metoprolol, moprolol, nadolol, nadoxolol, nebivalol, nipradilol, oxprenolol, perbutolol, pindolol, practolol, pronethalol, propranolol, sotalol, sufinalol, talindol, tertatolol, tilisolol, timolol, toliprolol, and xibenolol, and pharmaceutically acceptable salts or solvates thereof, or solvates of such salts.

In the present invention the betablocker is suitably metoprolol or atenolol or pharmaceutically acceptable salts or solvates thereof, or solvates of such salts. Particularly, the betablocker is metoprolol succinate (disclosed in U.S. Pat. No. 5,001,161), metoprolol tartrate or metoprolol fumarate.

The pure enantiomers, racemic mixtures and unequal mixtures of two enantiomers of a betablocker are within the scope of the present invention. It should also be understood that all the diastereomeric forms possible are within the scope of the invention.

In the present application, the term “betablocker” also include chemical modifications of the betablockers, such as esters, prodrugs and metabolites, whether active or inactive.

Cholesterol-Lowering Agents

The cholesterol-lowering agents referred to in this application include but are not limited to inhibitors of HMG-CoA reductase (3-hydroxy-3-methylglutaryl coenzyme A reductase). Suitably the HMG-CoA reductase inhibitor is a statin selected from the group consisting of atorvastatin, cerivastatin, fluvastatin, itavastatin, lovastatin, mevastatin, nicostatin, nivastatin, pravastatin and simvastatin, or a pharmaceutically acceptable salt, especially sodium or calcium, or a solvate thereof, or a solvate of such a salt. A particularly preferred statin is, however, a compound with the chemical name (E)-7-[4-(4-fluorophenyl)-6-isopropyl-2-[methyl(methylsulfonyl)-amino]-pyrimidin-5-yl](3R,5S)-3,5-dihydroxyhept-6-enoic acid, [also known as (E)-7-[4-(4-fluorophenyl)-6-isopropyl-2-[N-methyl-N-(methylsulfonyl)-amino]pyrimidin-5-yl](3R,5S)-3,5-dihydroxyhept-6-enoic acid] or a pharmaceutically acceptable salt or solvate thereof, or a solvate of such a salt. The compound (E)-7-[4-(4-fluorophenyl)-6-isopropyl-2-[methyl-(methylsulfonyl)-amino]-pyrimidin-5-yl](3R,5S)-3,5-dihydroxyhept-6-enoic acid, and its calcium and sodium salts are disclosed in European Patent Application, Publication No. EP-A-0521471, and in Bioorganic and Medicinal Chemistry, (1997), 5(2), 437-444. This latter statin is now known under its generic name rosuvastatin.

In the present application, the term “cholesterol-lowering agent” also includes chemical modifications of the HMG-CoA reductase inhibitors, such as esters, prodrugs and metabolites, whether active or inactive.

Pharmaceutical Formulations

The present invention relates to pharmaceutical compositions containing a betablocker and a cholesterol lowering agent as active ingredients.

Preferred combinations of a betablocker and a cholesterol-lowering agent are those where the betablocker is metoprolol or atenolol, particularly metoprolol succinate or metoprolol tartrate, and the cholesterol-lowering agent is a statin, particularly fluvastatin or a compound with the chemical name (E)-7-[4(4-fluorophenyl)-6-isopropyl-2-[methyl(methylsulfonyl)-amino]-pyrimidin-5-yl](3R,5S)-3,5-dihydroxyhept-6-enoic acid, or a pharmaceutically acceptable salt, especially the calcium and sodium salts, or solvate thereof, or a solvate of such a salt.

A most preferred combination comprises metoprolol, particularly metoprolol succinate or metoprolol tartrate, and (E)-7-[4-(4-fluorophenyl)-6-isopropyl-2-[methyl(methylsulfonyl)-amino]-pyrimidin-5-yl](3R,5S)-3,5-dihydroxyhept-6-enoic acid, or a pharmaceutically acceptable salt, especially the calcium and sodium salts, or solvate thereof, or a solvate of such a salt.

In the present invention, the formulation and/or kits of parts may comprise two or more betablockers in combination with a cholesterol-lowering agent, two or more cholesterol-lowering agents in combination with a betablocker or any combination thereof.

For clinical use, the betablocker and the cholesterol-lowering agent are formulated into a pharmaceutical formulation for oral, intravenous, subcutaneous, tracheal, bronchial, intranasal, pulmonary, transdermal, buccal, rectal, parenteral or some other mode of administration. The pharmaceutical formulation contains the betablocker and the cholesterol-lowering agent in admixture with a pharmaceutically acceptable adjuvant, diluent or carrier.

The total amount of active ingredients suitably is in the range of from about 0.1% (w/w) to about 95% (w/w) of the formulation, suitably from 0.5% to 50% (w/w) and particularly from 1% to 25% (w/w).

The molar ratio between the betablocker and the cholesterol-lowering agent may be in the range of from about 1000:1 to about 1:1000. The molar ratio between the betablocker and the cholesterol-lowering agent lies suitably, in the range of from 300:1 to 1:300, and particularly from 50:1 to 1:50.

In the preparation of the pharmaceutical formulations of the present invention the active ingredients may be mixed with solid, powdered ingredients, such as lactose, saccharose, sorbitol, mannitol, starch, amylopectin, cellulose derivatives, gelatin, or another suitable ingredient, as well as with disintegrating agents and lubricating agents such as magnesium stearate, calcium stearate, sodium stearyl fumarate and polyethylene glycol waxes. The mixture may then be processed into granules or pressed into tablets.

The active ingredients may be separately premixed with the other non-active ingredients, before being mixed to form a formulation. The active ingredients may also be mixed with each other, before being mixed with the non-active ingredients to form a formulation.

Soft gelatine capsules may be prepared with capsules containing a mixture of the active ingredients of the invention vegetable oil, fat, or other suitable vehicle for soft gelatine capsules. Hard gelatine capsules may contain granules of the active ingredients. Hard gelatine capsules may, also contain the active ingredients in combination with solid powdered ingredients such as lactose, saccharose, sorbitol, mannitol, potato starch, corn starch, amylopectin, cellulose derivatives or gelatine.

Dosage units for rectal administration may be prepared (i) in the form of suppositories which contain the active substance mixed with a neutral fat base; (ii) in the form of a gelatine rectal capsule which contains the active substance in a mixture with a vegetable oil, paraffin oil or other suitable vehicle for gelatine rectal capsules; (iii) in the form of a ready-made micro enema; or (iv) in the form of a dry micro enema formulation to be reconstituted in a suitable solvent just prior to administration.

Liquid preparations may be prepared in the form of syrups or suspensions, e.g. solutions or suspensions containing the active ingredients and the remainder consisting, for example, of sugar or sugar alcohols and a mixture of ethanol, water, glycerol, propylene glycol and polyethylene glycol. If desired, such liquid preparations may contain coloring agents, flavoring agents, preservatives, saccharine and carboxymethyl cellulose or other thickening agents. Liquid preparations may also be prepared in the form of a dry powder to be reconstituted with a suitable solvent prior to use.

Solutions for parenteral administration may be prepared as a solution of a formulation of the invention in a pharmaceutically acceptable solvent. These solutions may also contain stabilizing ingredients, preservatives and/or buffering ingredients. Solutions for parenteral administration may also be prepared as a dry preparation to by reconstituted with a suitable solvent before use.

The dose of the compounds to be administered will depend on the relevant indication, the age, weight and sex of the patient and may be determined by a physician. The dosage will particularly be in the range of from 0.01 mg/kg to 10 mg/kg.

The typical daily dose of the active ingredients varies within a wide range and will depend on various factors such as the relevant indication, the route of administration, the age, weight and sex of the patient and may be determined by a physician. In general, oral and parenteral dosages will be in the range of 0.1 to 1,000 mg per day of total active ingredients.

For the cholesterol-lowering agent, any dose used in clinical practice may be used in the formulations and kits of parts of the present invention.

In the present invention, “a therapeutically effective total amount” relates to a total amount of the a betablocker and the cholesterol-lowering agent which when given in combination gives a therapeutic effect, even though each amount when given separately may be less than the therapeutically effective amount.

Medical and Pharmaceutical Use

Also provided according to the present invention are formulations and kits of parts for use in medical therapy and particularly for use in the prophylactic or therapeutic treatment of atherosclerosis; the use of formulations of the present invention in the manufacture of medicaments for use in the prophylactic or therapeutic treatment of atherosclerosis, and methods of medical treatment or prophylaxis comprising the administration of a therapeutically effective total amount of a betablocker and a cholesterol-lowering agent to a patient suffering from, or susceptible to, atherosclerosis.

The betablocker and the cholesterol-lowering agent can be administered as a combined preparation for simultaneous, separate or sequential use in atherosclerosis therapy.

Furthermore, the betablocker can be administered prior to the administration of the cholesterol-lowering agent or vice versa.

The term ‘medical therapy’ as used herein is intended to include prophylactic, diagnostic and therapeutic regimens carried out in vivo or ex vivo on humans or other mammals.

The formulations of the invention are expected to be useful in prophylactic or therapeutic treatment of atherosclerosis, particularly in patients suffering from, or susceptible to hyperlipoproteinemia, and in particular hypercholesterolemia.

More particularly the formulations of the invention are expected to be useful in prevention of clinical events associated with the progression of atherosclerosis and/or acute vascular accidents related to atherosclerotic disease and plaque including but not limited to stroke, myocardial infarction (MI), congestive heart failure (CHF), cognitive decline, peripheral vascular disease, and renal dysfunction.

The formulations of the invention are furthermore expected to be useful in prophylactic or therapeutic treatment of cardiovascular complications in general, including, but not limited to, hypertension, diabetes mellitus, congestive heart failure (CHF), myocardial infarction (MI) including acute myocardial infarction (AMI), stroke, and mortality.

The following Example is intended to illustrate, but in no way limit the scope of the invention.

### EXAMPLE

A three-year placebo-controlled pilot study was designed to investigate the effect of the betablocker metoprolol on ultrasound-assessed atherosclerosis in patients with primary hypercholesterolemia on concomitant therapy with a cholesterol-lowering agent. More particularly, the trial was a prospective, randomised, double blind study, the betablocker was a controlled-release formulation of metoprolol succinate (metoprolol CR/XL) and the effect was assessed by measuring the intima-media thickness (IMT) in the carotid artery of the patients.

Intima-media thickness of the common carotid artery is commonly used as a surrogate variable for generalized atherosclerosis including coronary atherosclerosis (Wikstrand J, Wiklund O. Frontiers in cardiovascular science: Quantitative measurements of atherosclerotic manifestations in humans. Arterioscl Thromb. 1992; 12:114-119, and Grobbee D E, Bots M L. Carotid artery intima-media thickness as an indicator of generalized atherosclerosis. J. Int Med. 1994;, 236:567-573). Accordingly, IMT of the common carotid artery was selected as the major endpoint of the study. Good quality images of the far wall of the straight part of the common carotid artery are easy to obtain, and IMT can me measured in nearly all subjects with good reproducibility (Schmidt C, Wendelhag I. How can the variability in ultrasound measurement of intima-media thickness be reduced? Studies of interobserver variability in carotid and femoral arteries. Clin Phys. 1999;1:45-55.).

The inclusion criteria were willingness to participate, 20-70 years of age, serum cholesterol>6.5 mmol/L, low density lipid (LPL) cholesterol>5.0 mmol/L, and serum triglycerides<4.5 mmol/L.

Patients with hypercholesterolemia (n=129) were randomized at placebo-run run in, 62 to metoprolol CR/XL (AstraZeneca, Mölndal. Sweden) treatment (100 mg o.d.) and 67 to placebo. During the two-weeks of placebo run-in 15 subjects from the metoprolol CR/XL group and 11 subjects from the placebo group were withdrawn due to negative ultrasound examinations (n=3 and n=1, respectively), low total cholesterol, low LDL cholesterol or high triglyceride levels (n=6 and n=4, respectively), myocardial infarction (n=1 and n=0, respectively), nausea (n=1 and n=2, respectively), unwillingness to participate (n=4 and n=2, respectively) and other causes (n=0 and n=2, respectively).

After start of double-blind treatment 12 subjects from the metoprolol CR/XL group and 12 subjects from the placebo group were withdrawn due to myocardial infarction (n=2 and n=1, respectively), atrial fibrillation (n=2 and n=0, respectively), angina pectoris (n=1 and n=1, respectively), malignancy (n=1 and n=1, respectively), minor side effects (n=1 and n=5, respectively), given metoprolol CR/XL by other physician (n=0 and n=1, respectively), moved to other area (n=2 and n=0), compliance (n=1 and n=1, respectively) and unwillingness (n=2 and n=2, respectively).

Subsequently seventy-nine patients completed the study: 35 in the metoprolol CR/XL group and 44 in the placebo-group. The distribution of males and females in the groups were similar, 18 and 22 males and 17 and 22 females in the metoprolol CR/XL and placebo group, respectively. Likewise, the mean age was 60.6 and 59.6 for the metoprolol CR/XL and placebo group, respectively. The mean follow-up time was 2.97 years.

All analyses for the patients with hypercholesterolemia in the present example refers to the 79 subjects who completed the study. Two subjects in each group reduced the dose during follow-up from 100 mg to 50 mg (metoprolol CR/XL or placebo).

Total cholesterol, HDL cholesterol and heart rate decreased more in the metoprolol CR/XL group compared with the placebo group (p<0.05).

Cholesterol-Lowering Therapy in the Metoprolol CR/XL Group and the Placebo Group During Follow-Up

Subjects were treated with statins before inclusion, 31% in the metoprolol CR/XL group and 27% in the placebo group. However, all lipid-lowering drugs were withdrawn prior to start of double-blind medication.

The majority of subjects in the metoprolol CR/XL group and in the placebo group were treated with statins at the 1-year follow-up (91% and 80%, respectively), at the 2-year follow-up (88% and 82%, respectively), and at the 3-year follow up (94% and 84%, respectively). Ninety-four percent of the subjects in the metoprolol CR/XL group and 89% of the subjects in the placebo group were at any time during the 3-year follow-up treated with statins. Subjects were also treated with cholestyramine at the 1-year follow-up (23% and 30%, respectively), at the 2-year follow-up (26% and 23%, respectively), and at the 3-year follow-up (26% and 23%, respectively). All in the metoprolol CR/XL group and 96% of the subjects in the placebo group were treated with any lipid-lowering drug during follow-up.

During the 3-year follow-up LDL cholesterol was reduced by 44% in the metoprolol CR/XL group and by 38% in the placebo group (non-significant difference between groups).

Carotid Intima-Media Thickness (IMT) and Lumen Diameter in the Three Groups During Follow-Up

The intima-media thickness was defined as the distance between the leading edge of the lumen-intima interface of the far wall to the leading edge of the media-adventitia interface of the far wall. The lumen diameter was defined as the distance between the leading edges of the intima-lumen interface of the near wall and the lumen-intima interface of the far wall.

Mean values for common carotid IMT at the base-line examination and at the 3-year follow-up were 0.91±0.23 and 0.87±0.17 mm, respectively, in the metoprolol CR/XL group; and 0.89±0.18 and 0.92±0.17 mm, respectively, in the placebo group (p<0.05 between groups for difference in change during follow-up). The regression coefficient for the yearly change in common carotid IMT was −0.010±0.06 mm and +0.012±0.04 for the metoprolol CR/XL group and the placebo group, respectively; and the 95% CI for the difference between the placebo group and the metoprolol CR/XL group was 0.0004−0.0444 mm (p<0.05).

Mean values for carotid bulb IMT at the base-line examination and at the 3-year follow-up were 1.42±0.46 and 1.34±0.41 mm, respectively, in the metoprolol CR/XL group; and is 1.26±0.45 and 1.30±0.41 mm, respectively, in the placebo group (p=0.12 between groups for difference in change during follow-up).

Mean values for lumen diameter in the carotid artery at the base-line examination and at the 3-year follow-up were 6.30±0.78 and 6.15±0.85 mm, respectively, in the metoprolol CR/XL group; and 6.23±0.71 and 6.14±0.69 mm, respectively, in the placebo group (no significant differences between groups).

Mean values for baseline common carotid IMT in subjects that were withdrawn from double-blind medication during follow-up were 0.86±0.11 and 0.89±0.17 mm in the metoprolol CR/XL group and the placebo group, respectively (no significant difference between groups).

1. A pharmaceutical combination comprising a betablocker and an HMG-CoA reductase inhibitor wherein the betablocker is selected from the group consisting of: acebutolol, alprenolol, amosulalol, arotinolol, atenolol, befunolol, betaxolol, bevantolol, bisoprolol, bopindolol, bucumolol, bufetolol, bufuralol, bunitrolol, buprandolol, butofilolol, carazolol, carteolol, carvedilol, celiprolol, cetamolol, cloranolol, dilevalol, epanolol, indenolol, labetalol, levobunolol, mepindolol, metipranolol, metoprolol, moprolol, nadolol, nadoxolol, nebivalol, nipradilol, oxprenolol, perbutolol, pindolol, practolol, pronethalol, propranolol, sotalol, sufinalol, talindol, tertatolol, tilisolol, timolol, toliprolol, and xibenolol, and pharmaceutically acceptable salts or solvates thereof, or solvates of such salts; and the HMG-CoA reductase inhibitor is selected from the group consisting of: cerivastatin, itavastatin, lovastatin, mevastatin, nicostatin, nivastatin, and (E)-7-[4-(4-fluorophenyl)-6-isopropyl-2-[methyl(methylsulfonyl)-amino]-pyrimidin-5-yl](3R,5S)-3,5-dihydroxyhept-6-enoic acid or a pharmaceutically acceptable salt or solvate thereof, or a solvate of such a salt.

2. The pharmaceutical combination according to claim 1, wherein the betablocker is metoprolol or a pharmaceutically acceptable salt or solvate thereof, or a solvate of such a salt.

3. The pharmaceutical combination according to claim 2, wherein the betablocker is metoprolol succinate, metoprolol tartrate or metoprolol fumarate.

4. The pharmaceutical combination according to claim 1 or 2 wherein the HMG-CoA reductase inhibitor is (E)-7-[4-(4-fluorophenyl)-6-isopropyl-2-[methyl(methylsulfonyl)-amino]pyrimidin-5-yl](3R,5S)-3,5-dihydroxyhept-6-enoic acid or a pharmaceutically acceptable salt or solvate thereof, or a solvate of such a salt.

5. The pharmaceutical combination according to claim 1, wherein the molar ratio between the betablocker and the cholesterol-lowering agent lies in the range of from about 1000:1 to about 1:1000.

6. A pharmaceutical formulation comprising a betablocker and a cholesterol-lowering agent in admixture with a pharmaceutically acceptable adjuvant, diluent, or carrier.

7. A pharmaceutical formulation according to claim 6 wherein the betablocker is selected from the group consisting of: acebutolol, alprenolol, amosulalol, arotinolol, atenolol, befunolol, betaxolol, bevantolol, bisoprolol, bopindolol, bucumolol, bufetolol, bufuralol, bunitrolol, buprandolol, butofilolol, carazolol, carteolol, carvedilol, celiprolol, cetamolol, cloranolol, dilevalol, epanolol, indenolol, labetalol, levobunolol, mepindolol, metipranolol, metoprolol, moprolol, nadolol, nadoxolol, nebivalol, nipradilol, oxprenolol, perbutolol, pindolol, practolol, pronethalol, propranolol, sotalol, sufinalol, talindol, tertatolol, tilisolol, timolol, toliprolol, and xibenolol, and pharmaceutically acceptable salts or solvates thereof, or solvates of such salts.

8. The pharmaceutical formulation according to claim 7, wherein the betablocker is metoprolol or a pharmaceutically acceptable salt or solvate thereof, or a solvate of such a salt.

9. The pharmaceutical formulation according to claim 8, wherein the betablocker is metoprolol succinate, metoprolol tartrate, or metoprolol fumarate.

10. The pharmaceutical formulation according to any one of claims 6 to 8, wherein the cholesterol-lowering agent is an HMG-CoA reductase inhibitor.

11. The pharmaceutical formulation according to claim 10, wherein the HMG-CoA reductase inhibitor is selected from the group consisting of atorvastatin, cerivastatin, fluvastatin, itavastatin, lovastatin, mevastatin, nicostatin, nivastatin, pravastatin and simvastatin, or a pharmaceutically acceptable salt or solvate thereof, or a solvate of such a salt.

12. The pharmaceutical formulation according to claim 10, wherein the HMG-CoA reductase inhibitor is of (E)-7-[4-(4-fluorophenyl)-6-isopropyl-2-[methyl(methylsulfonyl)-amino]-pyrimidin-5-yl](3R,5S)-3,5-dihydroxyhept-6-enoic acid or a pharmaceutically acceptable salt or solvate thereof, or a solvate of such a salt.

13. The pharmaceutical formulation according to claim 6, wherein the molar ratio between the betablocker and the cholesterol-lowering agent lies in the range of from about 1000:1 to about 1:1000.

14. A kit of parts comprising:
(i) a vessel containing a betablocker and
(ii) a vessel containing (E)-7-[4-(4-fluorophenyl)-6-isopropyl-2-[methyl(methylsulfonyl)amino]-pyrimidin-5-yl](3R,5S)-3,5-dihydroxyhept-6-enoic acid or a pharmaceutically acceptable salt or solvate thereof, or a solvate of such a salt and instructions for the sequential, separate, or simultaneous administration of the betablocker and an HMG-CoA reductase inhibitor to a patient for which such administration is necessary or advantageous.

15. The kit of parts according to claim 14, wherein the betablocker is metoprolol or a pharmaceutically acceptable salt or solvate thereof, or a solvate of such a salt.

16. The kit of parts according to claim 15, wherein the betablocker is metoprolol succinate, metoprolol tartrate, or metoprolol fumarate.

17. The kit of parts according to claim 14, wherein the molar ratio between the betablocker and the cholesterol-lowering agent lies in the range of from about 1000:1 to about 1:1000.

18. A kit of parts comprising:
(i) a pharmaceutical formulation containing a betablocker in admixture with a pharmaceutically acceptable adjuvant, diluent, or carrier; and
(ii) a pharmaceutical formulation containing a cholesterol-lowering agent, in admixture with a pharmaceutically acceptable adjuvant, diluent, or carrier;
wherein the betablocker and the cholesterol-lowering agent are each provided in a form that is suitable for administration in conjunction with the other.

19. The kit of parts according to claim 18, comprising the betablocker and the cholesterol-lowering agent as a combined preparation for simultaneous, separate, or sequential use in atherosclerosis therapy.

20. The kit of parts according to claim 18 or 19, wherein the betablocker is metoprolol or a pharmaceutically acceptable salt or solvate thereof, or a solvate of such a salt.

21. The kit of parts according to claim 20, wherein the betablocker is metoprolol succinate, metoprolol tartrate, or metoprolol fumarate.

22. The kit of parts according to claim 18, wherein the cholesterol-lowering agent is an HMG-CoA reductase inhibitor.

23. The kit of parts according to claim 22, wherein the HMG-CoA reductase inhibitor is selected from the group consisting of atorvastatin, cerivastatin, fluvastatin, itavastatin, lovastatin, mevastatin, nicostatin, nivastatin, pravastatin and simvastatin, or a pharmaceutically acceptable salt or a solvate thereof, or a solvate of such a salt.

24. The kit of parts according to claim 22, wherein the HMG-CoA reductase inhibitor is of (E)-7-[4-(4-fluorophenyl)-6-isopropyl-2-[methyl(methylsulfonyl)amino]-pyrimidin-5-yl](3R,5S)-3,5-dihydroxyhept-6-enoic acid or a pharmaceutically acceptable salt or solvate thereof, or a solvate of such a salt.

25. The kit of parts according to claim 18, wherein the molar ratio between the betablocker and the cholesterol-lowering agent lies in the range of from about 1000:1 to about 1:1000.

26. A method for prophylactic or therapeutic treatment of a patient suffering from, or susceptible to, atherosclerosis, which method comprises administering to the patient a therapeutically effective total amount of
(i) a betablocker in admixture with a pharmaceutically acceptable adjuvant, diluent, or carrier; in conjunction with
(ii) a cholesterol-lowering agent in admixture with a pharmaceutically acceptable adjuvant, diluent, or carrier.

27. The method according to claim 26, wherein the administration of the betablocker and the cholesterol-lowering agent is simultaneous, separate, or sequential.

28. The method according to claim 26, wherein the betablocker is metoprolol or a pharmaceutically acceptable salt or solvate thereof, or a solvate of such a salt.

29. The method according to claim 27, wherein the betablocker is metoprolol or a pharmaceutically acceptable salt or solvate thereof, or a solvate of such a salt.

30. The method according to claim 28, wherein the betablocker is metoprolol succinate, metoprolol tartrate, or metoprolol fumarate.

31. The method according to claim 29, wherein the betablocker is metoprolol succinate, metoprolol tartrate, or metoprolol fumarate.

32. The method according to any one of claims 27 to 31, wherein the cholesterol-lowering agent is an HMG-CoA reductase inhibitor.

33. The method according to claim 32, wherein the HMG-CoA reductase inhibitor is (E)-7-[4-(4-fluorophenyl)-6-isopropyl-2-[methyl(methylsulfonyl)-amino]-pyrimidin-5-yl]-(3R,5S)-3,5-dihydroxyhept-6-enoic acid or a pharmaceutically acceptable salt or solvate thereof, or a solvate of such a salt.

34. The method according to claim 27, wherein the molar ratio between the betablocker and the cholesterol-lowering agent lies in the range of from about 1000:1 to about 1:1000.

35. A method for prophylactic or therapeutic treatment of a patient suffering from, or susceptible to, atherosclerosis which method comprises administering to the patient a formulation as defined in claim 6.

36. A method according to claim 35, wherein the patient suffers from, or is susceptible to, hypercholesterolemia or hyperlipoproteinemia.
